(19) United States
(12) Patent Application Publication
(10) Pub. No.: US 2026/0033989 A1
(43) Pub. Date: Feb. 5, 2026

(54) OPHTHALMIC SURGICAL HANDPIECES

(71) Applicant: Alcon Inc., Fribourg (CH)

(72) Inventors: Reto Grüebler, Greifensee (CH); Sean Christopher Madden, Mission Viejo, CA (US); Niccolo Maschio, Winterthur (CH)

(21) Appl. No.: 19/269,228

(22) Filed: Jul. 15, 2025

Related U.S. Application Data
(60) Provisional application No. 63/676,998, filed on Jul. 30, 2024.

(51) Int. Cl. A61F 9/007

(52) U.S. Cl. CPC A61F 9/00745

(57) ABSTRACT

In certain embodiments, a vitrectomy handpiece is provided. The vitrectomy handpiece includes a cutting assembly and a piezoelectric stack assembly. The piezoelectric stack assembly is coupled to the cutting assembly and is configured to actuate the cutting assembly to cut vitreous of an eye.

### INTRODUCTION

Microsurgical procedures frequently require precision cutting and/or removing of various body tissues. For example, certain ophthalmic surgical procedures may require cutting and removing portions of the vitreous humor, a transparent jelly-like material that fills the posterior segment of the eye. The vitreous humor, or vitreous, is composed of numerous microscopic fibrils that are often attached to the retina. Therefore, cutting and removing the vitreous may need to be done with great care to avoid traction on the retina, the separation of the retina from the choroid, a retinal tear, or, in the worst case, cutting and removal of the retina itself. In particular, delicate operations such as mobile tissue management (e.g., cutting and removal of vitreous near a detached portion of the retina or a retinal tear), vitreous base dissection, and cutting and removal of membranes may be particularly difficult.

When removing vitreous, ophthalmic surgeons typically utilize a vitrectomy handpiece to cut the vitreous into smaller fragments, and then suction the fragmented vitreous out of the eye. To cut the vitreous, an inner cutting member of the vitrectomy handpiece is actuated by a pneumatic chamber (and optionally a spring), and moved relative to a port of an outer cutting member. However, due to the design of traditional vitrectomy handpieces, and more particularly, the pneumatic chambers thereof, the efficacy of vitrectomy handpieces is greatly limited. For example, the pneumatic chamber may limit the cutting capabilities of a vitrectomy handpiece when the pressure in the chamber is too high, and/or flow through the chamber is restricted. Such restrictions reduce the effectiveness of the vitrectomy handpiece by interfering with the actuation of the inner cutting member and can potentially lead to incomplete tissue removal or other ophthalmic complications.

### BRIEF SUMMARY

The present disclosure relates generally to ophthalmic surgical instruments for performing ophthalmic surgical procedures, such as vitrectomy.

In certain embodiments, a vitrectomy handpiece is provided. The vitrectomy handpiece includes a handpiece comprising a cutting assembly and a piezoelectric stack assembly. The piezoelectric stack assembly is coupled to the cutting assembly and is configured to actuate the cutting assembly to cut vitreous of an eye.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A is an example of an ophthalmic surgical system that may be used to perform ophthalmic procedures on an eye, according to certain embodiments.

FIG. 1B illustrates example components of a surgical console of the ophthalmic surgical system of FIG. 1A, according to certain embodiments.

FIG. 2A is a side view of an example handpiece of the ophthalmic surgical system of FIGS. 1A-1B, according to certain embodiments.

FIG. 2B is a cross-sectional view of the handpiece of FIG. 2A, according to certain embodiments.

FIGS. 2C-2D illustrate various example distal end configurations of the handpiece shown in FIG. 2B, according to certain embodiments.

FIGS. 3A-3B illustrate cross-sectional views of an example piezoelectric stack assembly that may be used with the handpiece of FIG. 2A to achieve a longitudinal oscillatory motion, according to certain embodiments.

FIGS. 4A-4D illustrate cross-sectional views of additional example piezoelectric stack assemblies that may be used with the piezoelectric stack assembly shown in FIGS. 3A-3B to achieve a harmonic motion, according to certain embodiments.

FIG. 5 is a perspective view of an example piezoelectric stack assembly and a horn that may be used with the handpiece of FIG. 2A to achieve a harmonic motion, according to certain embodiments.

FIG. 6 is a frontal view of an angular piezoelectric stack assembly that may be used with the handpiece of FIG. 2A to achieve a harmonic motion, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

The vitreous body, often referred to as the vitreous humor or simply “the vitreous,” is a transparent, colorless, and gelatinous mass that fills the space between the lens and the retina of the eyeball (e.g., a posterior segment of the eye). The vitreous makes up about 80% of the volume of the eyeball and helps maintain the round shape of the eye. Additionally, the vitreous assists in absorbing external mechanical shocks to the eye, provides nutrients to the lens, and supports the retina. The vitreous is mostly comprised of water with trace amounts of collagen and hyaluronic acid, which provide the vitreous with its gelatinous structure.

Microsurgical cutting probes used in ophthalmic surgeries that involve removal of the vitreous may include a hollow outer cutting member, a hollow inner cutting member arranged coaxially with and movably disposed within the hollow outer cutting member, a port extending radially through the outer cutting member near the distal end of the outer cutting member, and a port extending radially through the inner cutting member near the distal end of the inner cutting member. Vitreous humor and/or membranes may be aspirated into the open port of the outer cutting member and the inner cutting member may be actuated to extend distally. As the inner cutting member extends distally, cutting surfaces on both the inner and outer cutting members may cooperate to cut the vitreous and/or membranes, and the cut tissue may then be aspirated away through the inner cutting member. Vitreous and/or membranes may then be aspirated into the open ports of both the outer and inner cutting members and the inner cutting member may be actuated to retract proximally. The inner and outer cutting members may cooperate to again cut vitreous and/or membranes and aspirate the cut tissue away.

To actuate the inner cutting member relative to the outer cutting member, conventional vitrectomy handpieces commonly utilize a pneumatic chamber which uses pressurized air to move the inner cutting member in two directions, or a pneumatic chamber which moves in one direction with a spring acting as a restoring force. However, use of the pneumatic chamber to move the inner cutting member limits the number of actuations, or the cut-rate of the vitrectomy handpiece because the pneumatic chamber can impose a certain amount of resistance to the movement of the inner cutting member. For example, if the pressure in the chamber is too high and/or the flow through the chamber is restricted, movement of the inner cutting member will be limited, reducing its effectiveness and potentially leading to incomplete tissue removal or other ophthalmic complications. As such, current vitrectomy handpieces present a variety of limitations.

Accordingly the embodiments described provide vitrectomy handpieces that provide greater precision and increased cut-rates, which thereby facilitate quicker, and more efficient removal of vitreous from an eye.

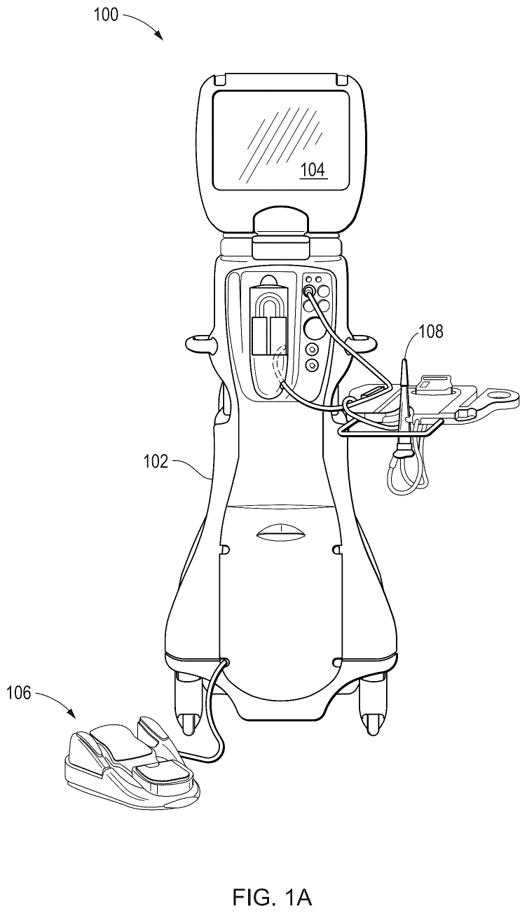

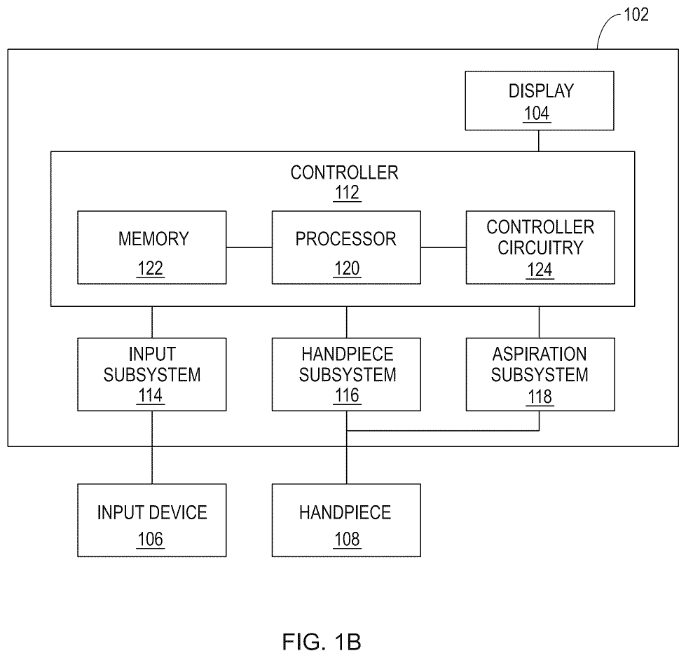

FIG. 1A is an example ophthalmic surgical system 100 that may be used to perform ophthalmic procedures on an eye, according to certain embodiments. The ophthalmic surgical system 100 includes a console 102 (also referred to as a “surgical console”), which includes a display 104, an input device 106 (e.g., a foot pedal), and a handpiece 108. The components of the ophthalmic surgical system 100 and the surgical console 102 are mechanically and/or electrically coupled as shown and described in more detail with reference to FIG. 1B.

FIG. 1B illustrates example components of the surgical console 102 of the ophthalmic surgical system 100 shown in FIG. 1A, according to certain embodiments. As shown, surgical console 102 includes a controller 112, an input subsystem 114, a handpiece subsystem 116, an aspiration subsystem 118, and a display 104. The controller 112 controls the operation of the surgical console 102 and is illustrated as being operationally coupled by a wired or wireless connection to the input device 106 via input subsystem 114, and to the handpiece 108 via handpiece subsystem 116 and aspiration subsystem 118. The controller 112 includes a processor 120, a memory 122, and controller circuitry 124.

Processor 120 may be any type of general purpose processor or could be a processor specifically designed for driving the subsystems illustrated in FIG. 1B, such as an application-specific integrated circuit (“ASIC”). The processor 120 may be, or include, a microprocessor, a microcontroller, an embedded microcontroller, a programmable digital signal processor, or any other programmable device operable to execute instructions stored in the memory 122 for operating surgical console 102. For example, the processor 120 may execute instructions in the memory 122 to receive inputs provided by input device 106 through input subsystem 114 and, in response, send instructions to the aspiration subsystem 118 and/or the handpiece subsystem 116 for operating the handpiece 108. Further, the processor 120 may execute instructions to generate a user interface view for display by display 104. In some instances, the processor 120 may also be or include a programmable gate array, programmable array logic, or any other device of combinations of devices operable to process electric signals.

Memory 122 can be any type of storage device or non-transitory computer-readable medium, such as random-access memory (“RAM”) or read-only memory (“ROM”), which is operable to receive, store, or recall data, including, but not limited to, electronic, magnetic, or optical memory, whether volatile or non-volatile. The memory 122 stores instructions executed by the processor 120. In example embodiments, functionality disclosed herein can be provided by the processor 120 and the memory 122 (i.e., software based), by the controller circuitry 124 (i.e., hardware based), or by a combination thereof. The memory 122 may include code stored thereon. The code may include instructions that may be executable by the processor 120. The code may be created, for example, using any programming language, including but not limited to, C, C++, Java, Python, Rust, or any other programming language (including assembly languages, hardware description languages, and database programming languages). In some instances, the code may be a program that, when executed by the processor 120, causes the surgical console 102 to operate subsystems 114, 116, and/or 118 for, e.g., driving the handpiece 108 or other devices in communication with the surgical console 102.

Handpiece 108 may be any suitable ophthalmic surgical instrument that can be operated on the basis of the embodiments described herein. For example handpiece 108 may be a vitrectomy handpiece (also referred to as a “vitrectomy probe”).

Handpiece subsystem 116 is configured to facilitate the operation of the handpiece 108. For example, handpiece subsystem 116 may control the operations (e.g., oscillatory motion) of a component (e.g., cutting assembly) of the handpiece 108. In particular, in one example, handpiece subsystem 116 applies voltage to a piezoelectric stack assembly (e.g., piezoelectric stack assembly 230) to cause an inner cutting member (e.g., inner cutting member 206) of the handpiece 108 to move in a longitudinal oscillatory motion, a rotational oscillatory motion, or a harmonic motion that is simultaneously a longitudinal oscillatory motion and a rotational oscillatory motion.

Aspiration subsystem 118 provides aspiration control for handpiece 108. In some embodiments, aspiration subsystem 118 may be operatively coupled to a surgical cassette during a surgical procedure. For example, the surgical cassette may be inserted into, attached to, and/or integrated with aspiration subsystem 118 via a coupling mechanism. When aspiration subsystem 118 is operatively coupled to a surgical cassette, aspiration subsystem 118 may control aspiration through the surgical cassette, which may in turn be coupled to the handpiece 108, for example, via a cable or other tether. In certain embodiments, the aspiration subsystem 118 includes one or more mechanical pumps having roller pump heads configured to engage with one or more corresponding pump assemblies on the surgical cassette. The engagement of the roller pump heads and pump assemblies generates a source of pressure and/or vacuum utilized during an ophthalmic surgical procedure.

Input device 106 may be any device that is capable of receiving commands from the user of the surgical console 102 in order to operate the handpiece 108 and/or other components of the surgical console 102. In FIG. 1A, input device 106 is illustrated as a foot pedal, however, other types of input devices are also within the scope of the disclosure. In one example, the user provides a command to the input device 106, which is received and relayed to the controller 112 by the input subsystem 114. In response, the controller 112 sends instructions to the handpiece subsystem 116 and/or aspiration subsystem 118 to control the operations of the handpiece 108 based on the user command.

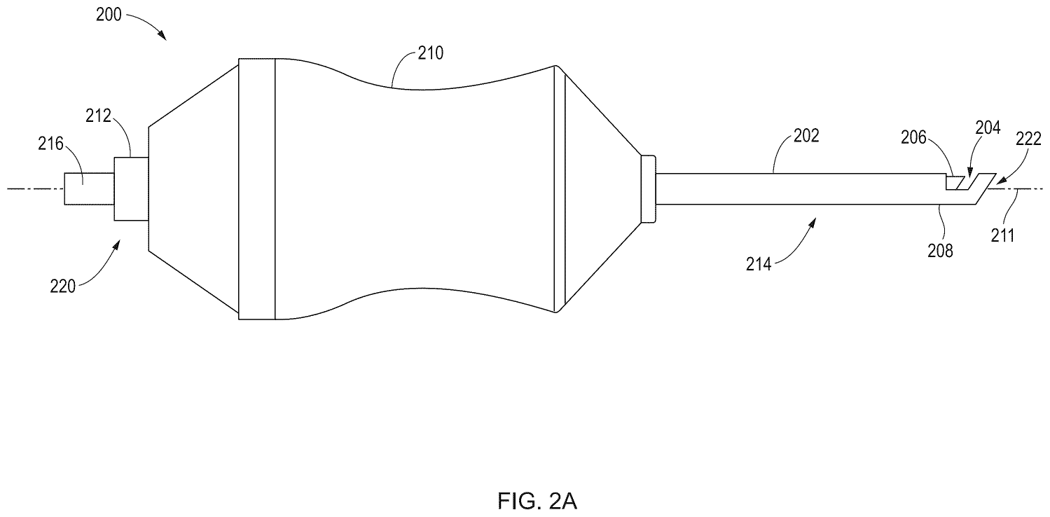

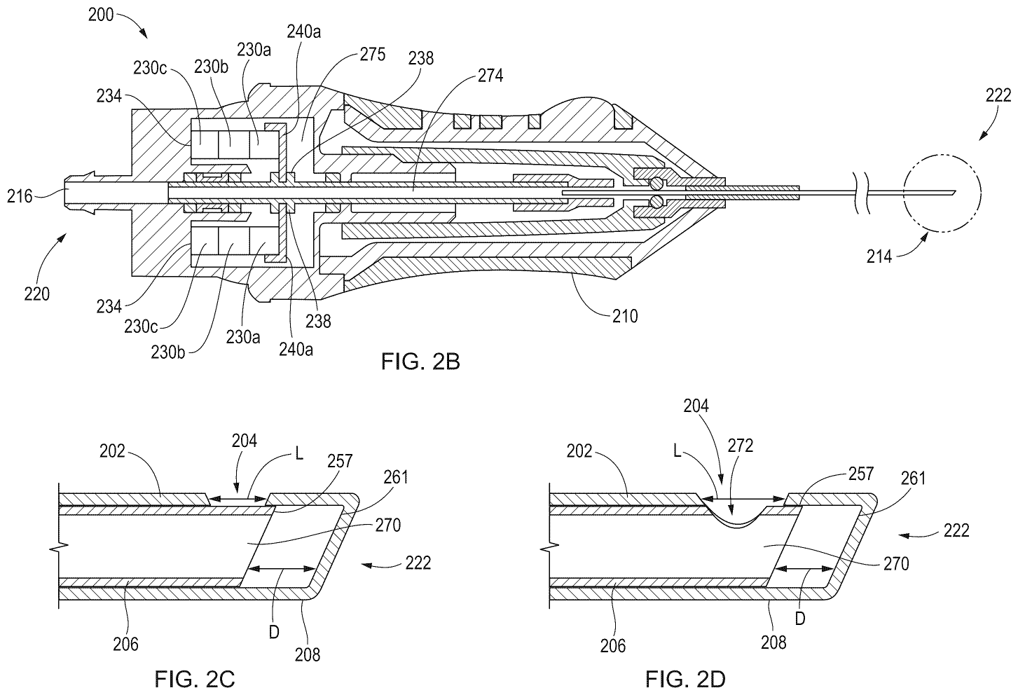

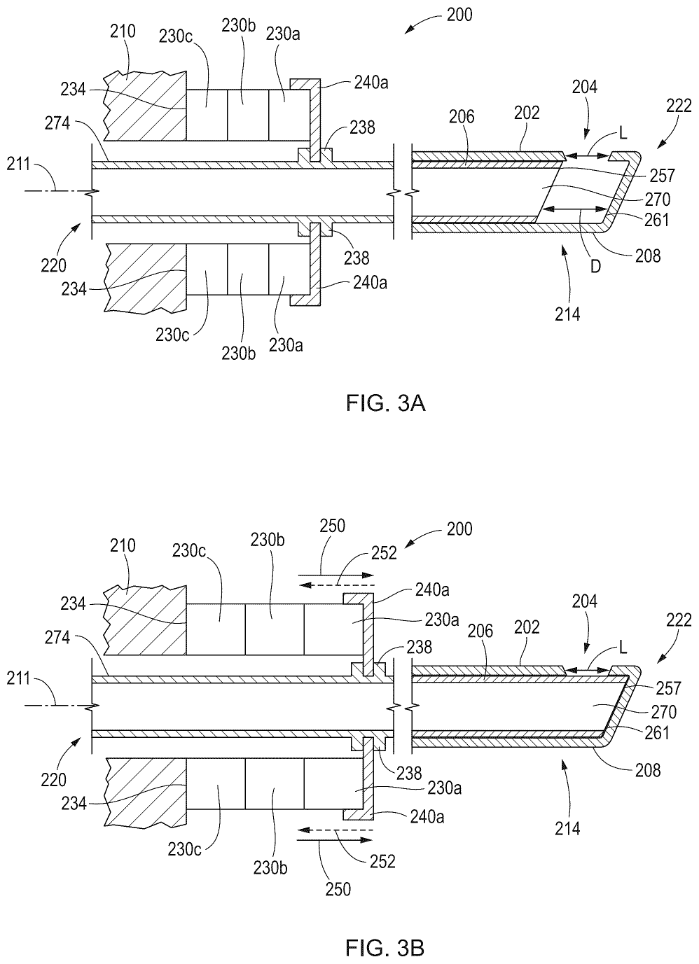

FIG. 2A is a side view of an example handpiece 200 of the ophthalmic surgical system 100 shown in FIGS. 1A-1B. The controller 112 is configured to control the handpiece 200. The handpiece 200 represents the handpiece 108 shown in FIGS. 1A-1B, and is a vitrectomy handpiece (sometimes referred to herein as a “vitrectomy probe”) which may be used, for example, to cut and/or remove vitreous from inside an eye.

The handpiece 200 includes an aspiration port 216 coupled to the handpiece 200 by a port adapter 212 at a proximal end 220. The aspiration port 216 provides ingress/egress for vacuum supply lines to be routed into an interior lumen of the handpiece 200. For example, the aspiration port 216 may provide a connection between the handpiece 200 and a vacuum supply line of a vacuum source within a surgical console (e.g., surgical console 102).

The handpiece 200 further includes a cutting assembly 214, which may be inserted into an eye, e.g., through a trocar cannula, for cutting the vitreous and other tissues and/or materials. The cutting assembly 214 of the handpiece 200 comprises a hollow, outer cutting member 202 (also known as a needle) with a first port 204 at a distal tip 208, and a hollow, inner cutting member 206 disposed within the outer cutting member 202. The outer cutting member 202 and the inner cutting member 206 of the cutting assembly 214 define a major (longitudinal) axis 211 of the handpiece 200.

The first port 204 of the outer cutting member 202 is configured to facilitate the provision of vacuum at a target tissue or material within a patient's eye for “grabbing” and manipulating the tissue or material during ophthalmic procedures. As shown, the distal tip 208 is beveled (e.g., angled) at an angle that is non-normal relative to the longitudinal axis 211 of the handpiece 200, thereby causing the first port 204 to have an elongated, e.g., ellipsoid shape. The beveled morphology of the distal tip 208 and thus, the elongated shape of the first port 204, increases the surface area of vacuum generation at first port 204 without requiring an increase in probe gauge. Accordingly, the handpiece 200 enables improved suction or “purchase” of ocular tissues/materials and thus, easier manipulation thereof, at smaller probe gauges. For clarity, enlarged cross-sectional views of distal tip 208 and first port 204 are illustrated in FIGS. 2C-2D, which are described in further detail below.

The inner cutting member 206 of the handpiece 200 may be formed of any material suitable for performing ophthalmic procedures. In certain embodiments, the inner cutting member 206 comprises a plastic or polymeric material. In such embodiments, a portion or substantially all of the inner cutting member 206 may be translucent or transparent. In certain other embodiments, the inner cutting member 206 comprises surgical-grade materials, such as aluminum, stainless steel (e.g., 316 or 316L stainless steel), or other alloys. In particular examples, the inner cutting member 206 is formed of Phynox, Elgiloy, or other suitable cobalt-chromium-nickel alloys. In particular examples, the inner cutting member 206 is formed of nitinol or other suitable nickel-titanium alloys. In further embodiments, the inner cutting member 206 may comprise a combination of metallic and polymeric materials.

A proximal end of the inner cutting member 206 may be directly or indirectly attached to a shaft 274 (seen in FIG. 2B) within an interior lumen of housing 210. In certain embodiments, the housing 210 is configured to be held by a user, such as a surgeon. For example, the housing 210 may be ergonomically contoured to substantially fit the hand of the user. In certain embodiments, the outer surface may be textured or have one or more gripping features formed thereon, such as one or more grooves and/or ridges. The housing 210 may be made from any materials commonly used for such instruments and suitable for ophthalmic surgery. For example, the housing 210 may be formed of a lightweight aluminum, a polymer, or other suitable material. In some embodiments, housing 210 may be sterilized and used in more than one surgical procedure, or may be a single-use device.

FIG. 2B is a cross-sectional view of the handpiece 200 of FIG. 2A, according to certain embodiments. FIGS. 2C-2D illustrate various example distal end configurations of the handpiece 200 shown in FIG. 2B, according to certain embodiments. Accordingly, FIGS. 2B-2D are described together herein for clarity purposes.

The handpiece 200 shown in FIG. 2B is driven by a plurality of piezoelectric disks 230a, 230b, 230c (230a-c) (collectively referred to herein as piezoelectric stack assembly 230) disposed in an enclosed drive chamber 275. The piezoelectric disks 230a-c comprise piezoelectric disks or rings that are stacked face-to-face and may be bonded together via, for example, epoxy or glass beads. Each of the piezoelectric disks 230a-c may be separately electrically coupled to the controller 112. Within the enclosed drive chamber 275, a proximal end of the piezoelectric stack assembly 230 abuts, and is attached (e.g., via an adhesive, ultrasonic welding, or other coupling mechanism) to a hard stop surface 234 of the housing 210, and a distal end of the piezoelectric stack assembly 230 abuts, and is attached to a moveable, anchor 240a. The anchor 240a is disposed over the distal end of the piezoelectric stack assembly 230 and within a first set of shaft grooves 238 laterally protruding from a shaft 274, which is coupled to the inner cutting member 206.

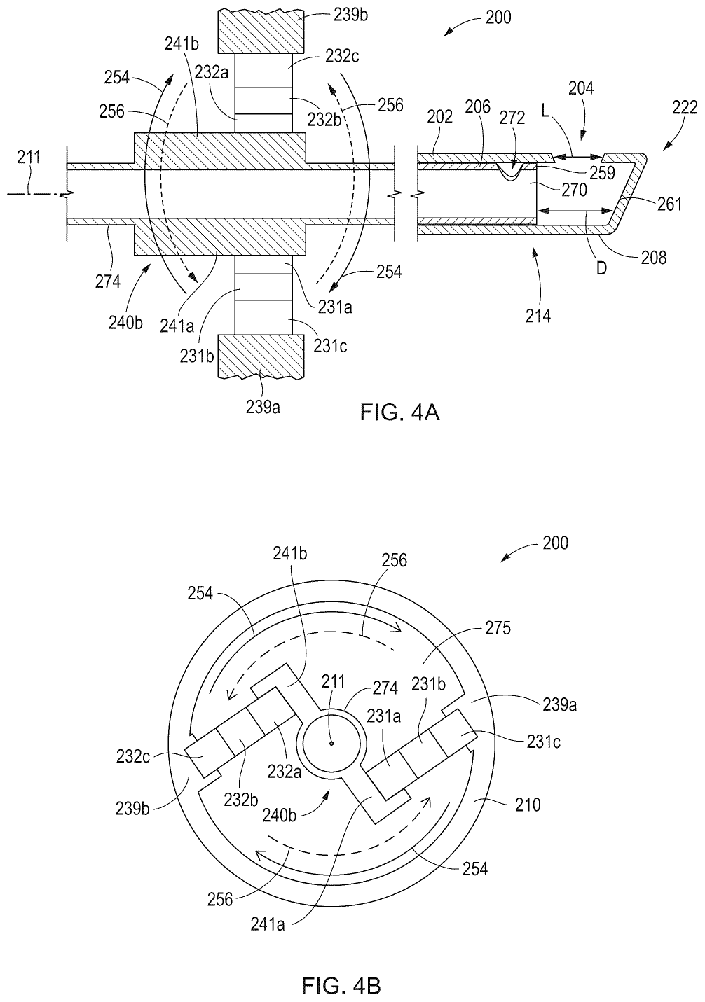

Generally, the inner cutting member 206 may oscillate within the outer cutting member 202 in response to movement of the shaft 274, which is caused by activation of one or more of the piezoelectric disks 230a-c. The activation of the piezoelectric disks 230a-c and various oscillatory movements of the inner cutting member 206 are described in further detail with reference to FIGS. 3A-3B, 4A-4D, 5, and 6.

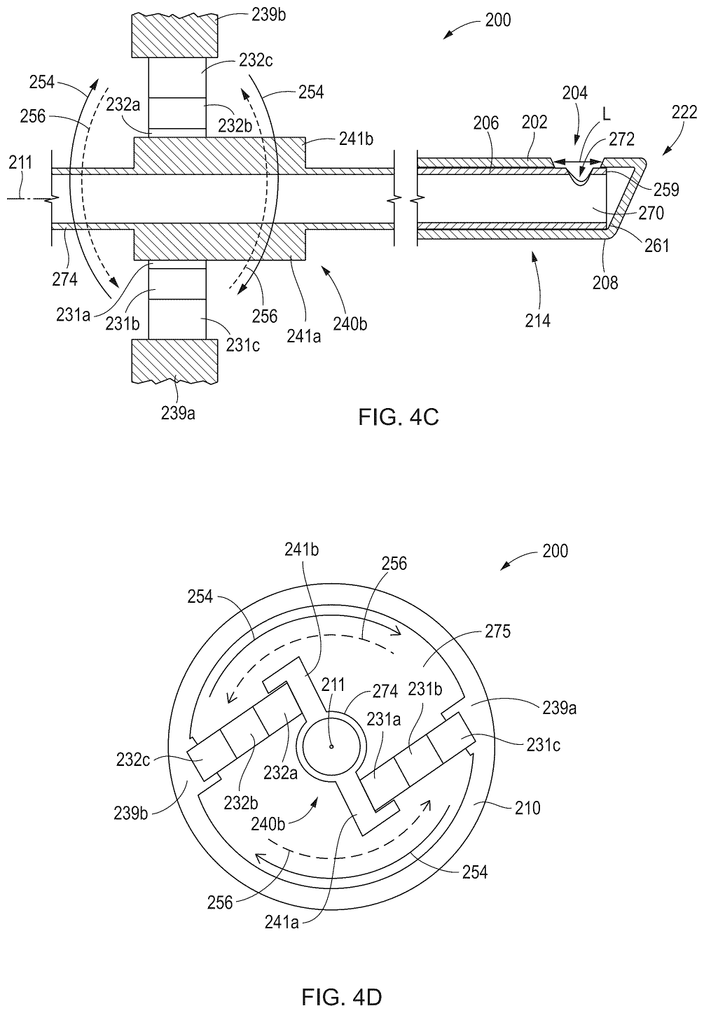

Note that FIG. 2B only illustrates one possible arrangement where a piezoelectric stack assembly is coupled to the shaft 274 to drive the inner cutting member 206, and that many other arrangements are also within the scope of the disclosure. For example, in certain embodiments, the handpiece 200 may be configured to include multiple piezoelectric stack assemblies that are each physically separated from each other along the longitudinal axis 211 of the handpiece 200, and each may form a separate assembly/packaging that may be coupled to the shaft 274 through different components. Such alternative embodiments are described in further detail with reference to FIGS. 4A-4D, 5, and 6.

Additionally or alternatively, the piezoelectric stack assembly 230 may include more than or less than three piezoelectric disks 230a, 230b, 230c. In certain embodiments, the piezoelectric stack assembly 230 may be coupled to the shaft 274 using more than one anchor (e.g., anchor 240a). Further, the anchor 240a may be coupled to the shaft 274 using other coupling mechanisms such as, a pin, a screw, an adhesive, etc.

Returning to FIGS. 2C-2D, the first port 204 of the outer cutting member 202 is defined by an opening with a length (L) that is between, for example, 1 millimeter (mm) and 10 mm (e.g., between 1.2 mm and 9.8 mm, 1.4 mm and 9.6 mm, 1.6 mm and 9.4 mm, or 1.8 mm and 9.2 mm). Additionally or alternatively, although shown as including one port 204 in FIGS. 2C-2D, the cutting assembly 214 may include more than one port in the outer cutting member 202.

The cutting assembly 214 also includes the distal tip 208 with a beveled (i.e., angled) end. The distal tip 208 of the outer cutting member 202 may be closed using, for example, spin closed machining, adhesive, welding (e.g., laser welding), etc. For example, the beveled end may be closed by laser welding a piece onto the angled end. Further, although shown as comprising a beveled end in FIGS. 2C-2D, the distal tip 208 may instead be flat.

As shown in FIGS. 2C-2D, the inner cutting member 206 has a beveled end similar in shape to the distal tip 208 of the outer cutting member 202, and is separated from the distal tip 208 by a distance (D). The distance between the inner cutting member 206 and the outer cutting member 202 changes when the inner cutting member 206 moves relative to an inner angled surface 261 of the outer cutting member 202.

Further, the inner cutting member 206 includes a cutting edge 257 and an open end at a distal port 270. The cutting edge 257 is configured to cut any vitreous material which may have been aspirated into the first port 204 of the outer cutting member 202. The distal port 270 is configured to facilitate aspiration of the vitreous material through the inner cutting member 206 and the shaft 274.

As shown in FIG. 2D, the inner cutting member 206 may also include a second port 272 that aligns with the first port 204 of the outer cutting member 202. The second port 272 is configured to facilitate additional cutting and/or aspiration of the vitreous material which enters through the first port 204.

In some embodiments, the handpiece 200 may include an aspiration opening near a distal end of the housing 210 to achieve aspiration. In such embodiments, material can be aspirated through the opening and flow through an aspiration tube (or line) that, at its proximal end, connects to the surgical console 102. For example, the aspiration tube may be proximally coupled to an aspiration source in the surgical console 102 and distally coupled to the opening from the exterior of the housing 210. In other words, the aspiration tube may be primarily disposed outside the handpiece 200.

FIGS. 3A-3B illustrate cross-sectional views of an example piezoelectric stack assembly 230 that may be used with the handpiece 200 of FIG. 2A to achieve a longitudinal oscillatory motion, according to certain embodiments. FIG. 3A illustrates a magnified cross-sectional view of the handpiece 200 in an open, first state, and FIG. 3B illustrates a magnified cross-sectional view of the handpiece 200 in a closed, second state. Accordingly, FIGS. 3A-3B are described together herein for clarity.

As illustrated in FIG. 3A, when the handpiece 200 is in the first state, the piezoelectric stack assembly 230 is in an inactivated, static state in which a voltage is not being applied to any of the piezoelectric disks 230a-c. Further, in the first state, the inner cutting member 206 is positioned behind the first port 204 of the outer cutting member 202. In other words, the cutting edge 257 is separated from the inner angled surface 261 of the outer cutting member 202 by a distance (D), such that there is a gap between the inner cutting member 206 and the outer cutting member 202.

When a voltage is applied to the piezoelectric stack assembly 230, the handpiece 200 transitions from the first state illustrated by FIG. 3A, to the second state illustrated by FIG. 3B. In other words, applying a voltage to the piezoelectric disks 230a-c activates the piezoelectric stack assembly 230 and causes piezoelectric crystals within the piezoelectric stack assembly 230 to at least partially deform. As the piezoelectric crystals deform, the piezoelectric stack assembly 230 extends (or moves) longitudinally in a first direction 250 towards the distal end 222 of the handpiece 200.

As illustrated by FIG. 3B, when the piezoelectric stack assembly 230 moves in the first direction 250, the piezoelectric stack assembly 230 extends from the hard stop surface 234 of the housing 210, and a distal end of the piezoelectric stack assembly 230 engages with the anchor 240a disposed in the first set of shaft grooves 238 of the shaft 274. Thus, the longitudinal movement of the piezoelectric stack assembly 230 results in longitudinal movement of the shaft 274 and the inner cutting member 206. Such movement displaces the inner cutting member 206 relative to the outer cutting member 202, thereby moving the cutting edge 257 in the distal direction and cutting tissue. The handpiece 200 is then in the closed, second state once the piezoelectric crystals within the piezoelectric disks 230a-c have completely or at least substantially deformed.

In the second state illustrated by FIG. 3B, the inner cutting member 206 extends past the first port 204 of the outer cutting member 202. For example, the cutting edge 257 of the inner cutting member 206 is in complete or almost complete contact with the inner angled surface 261 of the outer cutting member 202, thereby substantially eliminating the gap (shown by distance (D) in FIG. 3A) between the inner cutting member 206 and the outer cutting member 202. In other examples, the inner cutting member 206 may extend past the first port 204, but the cutting edge 257 may not contact the inner angled surface 261.

After the handpiece 200 has reached the second state shown in FIG. 3B, the handpiece 200 may return to the first state shown in FIG. 3A by discontinuing (or stopping) the voltage being applied to the piezoelectric stack assembly 230. When the voltage is no longer applied to the piezoelectric stack assembly 230, the piezoelectric crystals return to their initial, undeformed static state. As such, the piezoelectric stack assembly 230 recedes (or retracts), moving the inner cutting member 206 away from the distal end of the outer cutting member 202 in a second direction 252 that is opposite the first direction 250. Because the piezoelectric stack assembly 230 is configured to recede upon stoppage of the voltage, implementation of the piezoelectric stack assembly 230 eliminates the need for a spring within the handpiece 200.

Thus, the inner cutting member 206 of the cutting assembly 214 is configured to transition (or move) between the first state and second state shown in FIGS. 3A and 3B, respectively, by applying and terminating a voltage provided to the piezoelectric stack assembly 230. Such actuation of the cutting assembly 214 allows the inner cutting member 206 to achieve a longitudinal oscillatory motion.

In certain embodiments, the actuation of the cutting assembly 214 can be controlled by the piezoelectric stack assembly 230 to achieve a target cut-rate. For example, the piezoelectric stack assembly 230 can actuate the cutting assembly 214 to achieve a target cut-rate that can range from 1 cut per minute to greater than at least 50,000 cuts per minute (CPM), 60,000 CPM, 70,000 CPM, 80,000 CPM, 90,000 CPM, or 100,000 CPM.

The actuation of the cutting assembly 214 may involve modulating the inner cutting member 206 using an eigenfrequency, which may be defined as the natural frequency at which cutting assembly 214 tends to vibrate when it is disturbed from its position of equilibrium and then allowed to vibrate freely. When a system vibrates at its eigenfrequency, it experiences resonance, which can lead to large amplitude oscillations if the frequency of external forces matches the system's eigenfrequency. Using the eigenfrequency, an amplitude at which the cutting assembly 214 oscillates is elongated. In other words, the amplitude and therewith a maximum stroke distance of the cutting assembly 214 can be increased when the cutting assembly 214 oscillates at the eigenfrequency because excitation at eigenfrequency leads to resonance, and at resonance, amplitudes are much higher relative to oscillations not at the eigenfrequency.

As described above, the distance (D) travelled by the inner cutting member 206 of the cutting assembly 214 is controlled by activating the piezoelectric disks 230a-c. However, although FIG. 3B illustrates the activation of each of the piezoelectric disks 230a-c, in some embodiments, only one or less than all of the piezoelectric disks 230a, 230b, 230c may be activated. For example, only applying a voltage to piezoelectric disk 230a, but not piezoelectric disks 230b, 230c, results in the inner cutting member 206 partially moving in the first direction 250, such that only a portion of the distance (D) is travelled.

Additionally, flow of material into the handpiece 200 may be controlled through modulation of the inner cutting member 206 relative to the first port 204 during each oscillation of the inner cutting member 206. With each oscillation, the inner cutting member 206 transitions from its position in the first state shown in FIG. 3A to its position in the second state shown in FIG. 3B and then back to its position in the first state. Therefore, the amount of material aspirated through the first port 204 during each oscillation can be controlled by modulating access to the first port 204.

As an example, during each oscillation, the amount of material that flows into the handpiece 200 can be decreased by activating the piezoelectric disks 230a-c in a manner that causes the inner cutting member 206 to effectively reduce the size of the opening provided by the first port 204. For example, in the inner cutting member 206's first state, instead of being positioned at a distance of (D) relative to the outer cutting member 202 (as shown in FIG. 3A), the inner cutting member 206 can be positioned at a distance of less than (D) relative to the outer cutting member 202, thereby effectively partially blocking the first port 204. In such an example, the inner cutting member 206 oscillates from the first position where the diameter of the opening of the first port 204 is less than (L) to a second state and back to the first state. Further, during certain periods throughout the operation where the inner cutting member 206 is not being used for cutting, and thereby not oscillating, the inner cutting member 206 may be held in a position which partially blocks the first port 204, effectively reducing the size of the opening provided by the first port 204. As a result, the flow of material into the first port 204 can be reduced relative to holding the inner cutting member 206 in a fully retracted state where the first port 204 is not blocked.

As another example, the rate at which material flows into the handpiece 200 can be decreased by maintaining activation of the piezoelectric disks 230a-c for a longer duration of time during each oscillation of the inner cutting member 206. As discussed above, with each oscillation, the inner cutting member 206 transitions from its position in the first state shown in FIG. 3A to its position in the second state shown in FIG. 3B and then back to its position in the first state. During each oscillation, the inner cutting member 206 is configured to remain in the second state for a particular duration of time. Therefore, by increasing the duration of time the inner cutting member 206 is configured to remain in the second state during each oscillation, the rate at which material flows into the handpiece 200 will be reduced because material can be aspirated through the first port 204 for a shorter duration of time. Alternatively, by decreasing the duration of time the inner cutting member 206 is configured to remain in the second state during each oscillation, the rate at which material flows into the handpiece 200 will be increased because material can be aspirated through the first port 204 for a longer duration of time.

In some embodiments, instead of the piezoelectric disks 230a-c, the piezoelectric stack assembly 230 comprises a plurality of piezoelectric cones. The piezoelectric cones may be configured to transition between a conical shape and a disk-like shape. The piezoelectric cones may maintain the disk-like shape when in an inactivated state, and transition to the conical shape when in an activated state. The piezoelectric cones may be implemented similarly to the piezoelectric disks 230a-c, but less force may be used by the piezoelectric cones to achieve similar or greater displacement of the inner cutting member 206.

Note that although FIGS. 3A and 3B are described with reference to the handpiece 200 shown in FIGS. 2A-2B, the embodiments of FIGS. 3A and 3B are applicable to other types of handpieces.

FIGS. 4A-4D illustrate cross-sectional views of additional example piezoelectric stack assemblies 231, 232 that are configured to be used in conjunction with the piezoelectric stack assembly 230 shown in FIGS. 3A-3B to achieve a harmonic motion, according to certain embodiments. FIG. 4A illustrates a magnified cross-sectional view of the handpiece 200 in a non-rotated, first state, and FIG. 4B illustrates a top-down view of the handpiece 200 in the non-rotated, first state. FIG. 4C illustrates a magnified cross-sectional view of the handpiece 200 in a rotated, second state, and FIG. 4D illustrates a top-down view of the handpiece 200 in the rotated, second state. Accordingly, FIGS. 4A-4D are described together herein for clarity purposes.

In addition to piezoelectric stack assembly 230, in the embodiments of FIG. 4A, the handpiece 200 includes a second piezoelectric stack assembly 231 and a third piezoelectric stack assembly 232 (collectively referred to herein as additional piezoelectric stack assemblies 231, 232) that are configured to provide a rotational oscillatory motion to the cutting assembly 214. The addition of the rotational oscillatory motion to the longitudinal oscillatory motion provided by the piezoelectric stack assembly 230, as discussed with reference to FIGS. 3A-3B, allows the cutting assembly 214 to achieve a harmonic motion. The harmonic motion is simultaneously a longitudinal oscillatory motion and a rotational oscillatory motion.

The second piezoelectric stack assembly 231 includes three piezoelectric elements 231a, 231b, 231c (231a-c), and the third piezoelectric stack assembly 232 includes three piezoelectric elements 232a, 232b, 232c (232a-c). The piezoelectric elements included in the piezoelectric stack assemblies 231, 232 include piezoelectric chips or plates that are stacked face-to-face and may be bonded together via epoxy or glass beads.

In certain embodiments, when implemented in the handpiece 200 shown in FIG. 2A, the additional piezoelectric stack assemblies 231, 232 may be disposed proximally or distally in a second enclosed drive chamber 275b along the longitudinal axis 211 relative to the piezoelectric stack assembly 230. As seen in FIGS. 4A-4D, the additional piezoelectric stack assemblies 231, 232 are disposed between a moveable, second anchor 240b and a set of housing grooves 239a, 239b (239a-b), orthogonal to the longitudinal axis 211. The second anchor 240b includes arms 241a, 241b (241a-b) which protrude laterally outwards from the shaft 274 and extend longitudinally along the shaft 274 (best seen in FIG. 4A), and the set of housing grooves 239a-b protrude laterally inwards towards the shaft 274. Thus, outer ends of the piezoelectric stack assemblies 231, 232 abut, and are attached (e.g., via an adhesive, ultrasonic welding, or other coupling mechanism) to the set of housing grooves 239a-b, and inner ends of the piezoelectric stack assemblies 231, 232 abut, and are attached (e.g., via an adhesive, ultrasonic welding, or other coupling mechanism) to the arms 241a-b. The set of housing grooves 239a-b are disposed over the outer ends of the piezoelectric stack assemblies 231, 232, and the arms 241a-b are disposed over the inner ends of the piezoelectric stack assemblies 231, 232.

In the first state illustrated by FIGS. 4A-4B, the piezoelectric stack assemblies 231, 232 are in an inactivated, static state in which a voltage is not being applied to any of the piezoelectric elements 231a-c, 232a-c. When the handpiece 200 is in the first state, the flat cutting edge 259 of the inner cutting member 206 is separated from the inner angled surface 261 of the outer cutting member 202 by the distance (D), such that there is a gap between the inner cutting member 206 and the outer cutting member 202. Additionally, the piezoelectric stack assemblies 231, 232 are disposed at a proximal end of the second anchor 240b (best seen in FIG. 4A).

When a voltage is applied to the piezoelectric stack assemblies 231, 232, the handpiece 200 transitions from the first state illustrated by FIGS. 4A-4B, to the second state illustrated by FIG. 4C-4D. In other words, applying a voltage to the piezoelectric stack assemblies 231, 232 activates the piezoelectric stack assemblies 231, 232 and causes piezoelectric crystals within the piezoelectric stack assemblies 231, 232 to at least partially deform. As the piezoelectric crystals deform, the piezoelectric stack assemblies 231, 232 extend (or move) longitudinally, but lateral to the longitudinal axis 211, causing rotation of the shaft 274 in a first rotational direction 254. The rotation of the shaft 274 occurs simultaneously with the linear movement caused by the piezoelectric stack assembly 230 described with reference to FIGS. 3A-3B. Thus, as the shaft 274 rotates, it also moves towards the distal end 222 and slides relative to the piezoelectric stack assemblies 231, 232.

As illustrated by FIGS. 4C-4D, when the piezoelectric stack assemblies 231, 232 move in the first rotational direction 254, the piezoelectric stack assemblies 231, 232 extend from the set of housing grooves 239a-b, and the inner ends of the piezoelectric stack assemblies 231, 232 engage with the arms 241a-b of the second anchor 240b. Thus, the longitudinal movement of the arms 241a-b results in rotational movement of the shaft 274 and the inner cutting member 206. Such movement of the inner cutting member 206 displaces the inner cutting member 206 relative to the outer cutting member 202, thereby moving the flat cutting edge 259 in the first rotational direction 254 and cutting tissue. The handpiece 200 is then in the closed, second state once the piezoelectric crystals within the piezoelectric stack assemblies 231, 232 have completely or at least substantially deformed.

In the second state illustrated by FIGS. 4C-4D, the inner cutting member 206 extends past the first port 204 of the outer cutting member 202, and the flat cutting edge 259 has at least partially rotated relative to the first port 204. That is, the flat cutting edge 259 at least partially contacts the inner angled surface 261 of the outer cutting member 202, thereby substantially eliminating the gap (shown by distance (D) in FIG. 4A) between the inner cutting member 206 and the outer cutting member 202.

After the handpiece 200 has reached the second state shown in FIGS. 4C-4D, the handpiece 200 may return to the first state shown in FIGS. 4A-4B by discontinuing (or stopping) the voltage being applied to the piezoelectric stack assemblies 231, 232. When the voltage is no longer being applied to the piezoelectric stack assemblies 231, 232, the piezoelectric crystals return to their initial, undeformed state. As such, the piezoelectric stack assemblies 231, 232 recede (or retract) while also rotating the shaft 274 back in a second rotational direction 256 that is opposite the first rotational direction 254. As an example, the first rotational direction 254 is a clockwise direction, and the second rotational direction 256 is a counter-clockwise direction.

Thus, the inner cutting member 206 of the cutting assembly 214 is configured to transition (or move) between the first state and second state shown in FIGS. 4A-4B and 4C-4D, respectively, by applying and terminating a voltage provided to the piezoelectric stack assemblies 230, 231, 232. Such actuation of the cutting assembly 214 allows the inner cutting member 206 to achieve a harmonic motion that is simultaneously a longitudinal oscillatory motion and a rotational oscillatory direction. Actuation of the cutting assembly 214 using the harmonic motion may further reduce a port to tip distance (e.g., distance travelled by flat cutting edge 259 relative to port 204).

Although FIG. 4D illustrates the activation of each of the piezoelectric elements 231a-c, 232a-c, in some embodiments, only one or more of the piezoelectric elements 231a-c, 232a-c may be activated. For example, only applying a voltage to piezoelectric elements 231a, 232a, but not piezoelectric disks 231b-c, 232b-c, results in the inner cutting member 206 partially rotating in the first rotational direction 254.

In some embodiments, instead of utilizing the longitudinal oscillatory motion provided by the piezoelectric stack assembly 230, the handpiece 200 may instead cut the material entering the first port 204 using only the rotational oscillatory motion provided by the additional piezoelectric stack assemblies 231, 232. That is, instead of longitudinally oscillating the inner cutting member 206, the inner cutting member 206 may be kept in the second state shown in FIG. 4C by maintaining activation of the piezoelectric stack assembly 230. In the second state, the flat cutting edge is kept in contact with or positioned very close to the inner angled surface 261. While the flat cutting edge is in the second state, the additional piezoelectric stack assemblies 231, 232 can be activated to rotationally oscillate the inner cutting member 206, thereby rotating the second port 272 relative to the first port 204. Such rotational movement of the second port 272 results in the cutting of material entering the first port 204.

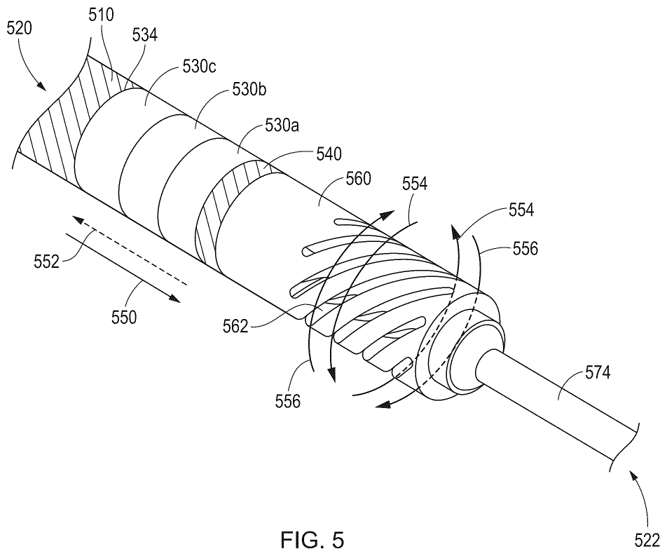

FIG. 5 is a perspective view of an example piezoelectric stack assembly 530 and a horn 560 that may be used with the handpiece 200 of FIG. 2A to achieve a harmonic motion, according to certain embodiments. The handpiece 200 is shown in FIG. 5 with portions of the housing 510 removed so that components of the handpiece 200 may be viewed more clearly. The piezoelectric stack assembly 530 and the horn 560 may be used instead of the piezoelectric stack assembly 230 configuration shown in FIGS. 2B-2D.

The piezoelectric stack assembly 530 seen in FIG. 5 includes a plurality of piezoelectric disks 530a, 530b, 530c (530a-c) disposed between a fixed, hard stop surface 534 of the housing 510 and a moveable, first anchor 540. The hard stop surface 534 may be similar to the hard stop surface 234, and the first anchor 540 may be similar to the anchor 240a, as described with reference to FIGS. 2B and 3A-3B. That is, a proximal end of the piezoelectric stack assembly 530 abuts, and is attached (e.g., via an adhesive, ultrasonic welding, or other coupling mechanism) to the hard stop surface 534, and a distal end of the piezoelectric stack assembly 530 abuts, and is attached to the first anchor 540. The first anchor 540 is further coupled to a proximal end of the horn 560.

The piezoelectric stack assembly 530 operates similar to the piezoelectric stack assembly 230 described with reference to FIGS. 3A-3B. That is, the piezoelectric stack assembly 530 is configured to produce a longitudinal movement in a first direction 550 when a voltage is applied, and a longitudinal movement in a second direction 552 when the voltage is no longer applied. When the piezoelectric stack assembly 530 moves in the first direction 550, the piezoelectric stack assembly 530 extends distally from the hard stop surface 534, and a distal end of the piezoelectric stack assembly 530 engages the first anchor 540, pushing against the horn 560. When the piezoelectric stack assembly 530 moves in the second direction 552, the piezoelectric stack assembly 530 recedes proximally towards the hard stop surface 534, pulling back the first anchor 540 and the horn 560 to their original positions.

The horn 560 may be made from a titanium alloy and comprises a plurality of helical slits 562. A distal end 522 of the horn 560 is attached to a shaft 274, which may be coupled to the inner cutting member 206 similar to as described with reference to FIG. 2B. The slits 562 may comprise a width that is between 2% and 65% of the outside diameter of the horn 560. This, of course, will affect how many slits can be made on the horn 560 (e.g., if slits 562 are 65% of the diameter of the horn 560, then only one slit 562 may be cut into the horn 560). The selected width of slits 562 will depend upon the desired amount of torsional movement. The depth of the slits 562 in the horn 560 is preferably between 4% and 45% of the outside diameter of horn 560. The slits 562 comprise hollow grooves or openings, but may also have a flat or square cut bottom, or a rounded or radiused bottom. The length of the slits 562 may be between 8% and 75% of the length of the larger diameter of the horn 560. The pitch of slits 562 may be between 125% and 500% of the larger diameter of the horn 560. By way of example, one suitable configuration of the slits 562 on the horn 560 with an outside diameter of 0.475 inches is a total of eight slits 562, having a width of 0.04 inches, a length of 0.7 inches, and a pitch of 1.35 inches gives suitable rotational movement of the horn 560 without compromising the longitudinal movement of the horn 560.

The helical slits 562 are configured to introduce a rotational oscillatory motion, caused by the longitudinal oscillatory motion from the piezoelectric stack assembly 530. For example, when the piezoelectric stack assembly 530 moves longitudinally in the first direction 550, the piezoelectric stack assembly 530 pushes against the horn 560, and the slits 562 cause a distal end 522 of the horn 560 to rotate relative to its proximal end 520. As such, the distal end 522 of the horn 560 moves in a first rotational direction 554 when the piezoelectric stack assembly 530 is activated (i.e., a voltage is applied). When the piezoelectric stack assembly 530 is deactivated (i.e., no voltage is applied), the horn 560 moves back in a second rotational direction 556. Thus, the simultaneous combination of longitudinal oscillatory motion produced by the piezoelectric stack assembly 530 and the rotational oscillatory motion produced by the horn 560 results in a harmonic motion of the shaft 574.

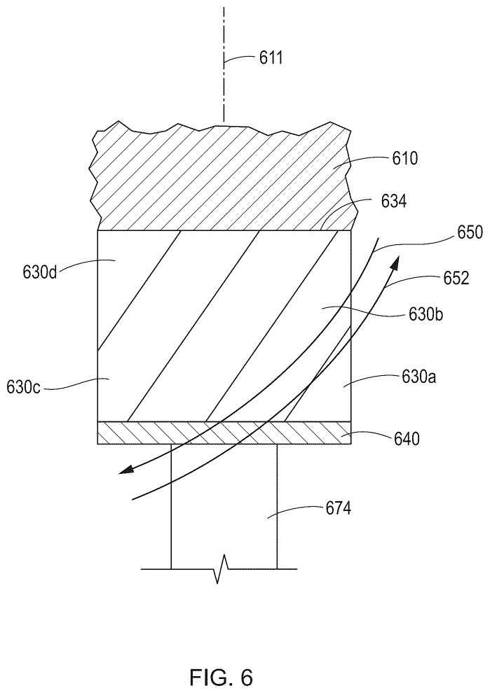

FIG. 6 is a frontal view of an angular piezoelectric stack assembly 630 that may be used with the handpiece 200 of FIG. 2A to achieve a harmonic motion, according to certain embodiments. The handpiece 200 is shown in FIG. 6 with portions of the housing 610 removed so that components of the handpiece 200 may be viewed more clearly. The angular piezoelectric stack assembly 630 may be used instead of the piezoelectric stack assembly 230 configuration shown in FIGS. 2B-2D.

As seen in FIG. 6, the angular piezoelectric stack assembly 630 includes a plurality of angular piezoelectric elements 630a, 630b, 630c, 630d (630a-d) with proximal and distal ends which have been cut at approximately 45° (degrees) (±30°). The plurality of angular piezoelectric elements 630a-d are disposed circumferentially about a longitudinal axis 611 of the handpiece 200. Additionally, the angular piezoelectric stack assembly 630 is disposed between a fixed, hard stop surface 634 of the housing 610 and a moveable, first anchor 640. The hard stop surface 634 may be similar to the hard stop surface 234, and the first anchor 640 may be similar to the anchor 240a as both described with reference to FIGS. 2B and 3A-3B. That is, a proximal end of the angular piezoelectric elements 630a-d abut, and are attached (e.g., via an adhesive, ultrasonic welding, or other coupling mechanism) to the hard stop surface 634, and a distal end of the angular piezoelectric elements 630a-d abuts, and is attached to the first anchor 640. The first anchor 640 is further coupled to the shaft 674 similar to as described with reference to FIG. 2B.

Due to the diagonal (e.g., approximately 45°) cuts at the ends of the angular piezoelectric elements 630a-d, the angular piezoelectric stack assembly 630 is configured to generate a harmonic motion (e.g., a simultaneous longitudinal and rotational motion) when activated. That is, when a voltage is applied, the angular piezoelectric stack assembly 630 moves in a first direction 650, and when the voltage is no longer applied, the angular piezoelectric stack assembly 630 returns to its initial, static state by moving in a second direction 652. In response to the harmonic motion of the angular piezoelectric stack assembly 630, the shaft 674 and an inner cutting member (e.g., inner cutting member 206) coupled thereto also move in the harmonic motion.

In certain embodiments, the angular piezoelectric elements 630a-d include piezoelectric chips or plates that are stacked face-to-face and may be bonded together via epoxy or glass beads. The angular piezoelectric stack assembly 630 may also include more or less than four angular piezoelectric elements. Further, the angular piezoelectric elements 630a-d may be arranged at different angles relative to the longitudinal axis 611.

In some embodiments, a vitrectomy handpiece comprises a handpiece including a piezoelectric stack assembly, and a cutting assembly configured to be coupled to the handpiece. The piezoelectric stack assembly is configured to actuate the cutting assembly to cut vitreous of an eye. The cutting assembly may be a single use cutting assembly, which may only be used in a single procedure. The handpiece may be configured to be reused with a second cutting assembly. As an example, the handpiece is coupled to the cutting assembly by a bayonet catch, a thread, a ball lock, or other similar coupling mechanism.

The use of the piezoelectric stack assembly to actuate a cutting assembly, as described herein, provides various improvements over conventional handpieces which may utilize a pneumatic chamber to actuate the cutting assembly. For example, the piezoelectric stack assembly enables a handpiece to achieve greater cut-rates and greater control over each individual cut, whereas the cut-rates and control of conventional handpieces are limited by the flow of air through the pneumatic chamber. As a result of greater control and increased cut-rates provided by the piezoelectric stack assembly, the handpiece experiences improved precision and efficiency when removing vitreous from the eye.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

### EXAMPLE EMBODIMENTS

Embodiment 1: A vitrectomy handpiece, the vitrectomy handpiece comprising: a handpiece comprising: a cutting assembly; and a piezoelectric stack assembly coupled to the cutting assembly and configured to: actuate the cutting assembly to cut vitreous of an eye, wherein the actuation of the cutting assembly comprises: moving an inner cutting member of the cutting assembly in a longitudinal oscillatory motion.

Embodiment 2: A vitrectomy handpiece, the vitrectomy handpiece comprising: a handpiece comprising: a cutting assembly; and a piezoelectric stack assembly coupled to the cutting assembly and configured to: actuate the cutting assembly to cut vitreous of an eye, wherein the actuation of the cutting assembly comprises: moving an inner cutting member of the cutting assembly in a harmonic motion, and wherein the harmonic motion comprises a motion that is simultaneously a longitudinal oscillatory motion and a rotational oscillatory motion.

Embodiment 3: A vitrectomy handpiece, the vitrectomy handpiece comprising: a handpiece comprising a piezoelectric stack assembly; and a cutting assembly configured to be coupled to the handpiece, wherein the piezoelectric stack assembly is configured to actuate the cutting assembly to cut vitreous of an eye.

Embodiment 4: The vitrectomy handpiece of Embodiment 3, wherein the handpiece is configured to be reused with a second cutting assembly.

Embodiment 5: The vitrectomy handpiece of Embodiment 3, wherein the cutting assembly is a single use cutting assembly.

Embodiment 6: The vitrectomy handpiece of Embodiment 3, wherein the handpiece is coupled to the cutting assembly by a bayonet catch.

Embodiment 7: The vitrectomy handpiece of Embodiment 3, wherein the handpiece is coupled to the cutting assembly by a thread.

Embodiment 8: The vitrectomy handpiece of Embodiment 3, wherein the handpiece is coupled to the cutting assembly by a ball lock.

What is claimed is:

1. A vitrectomy handpiece, the vitrectomy handpiece comprising:
a handpiece comprising:
a cutting assembly; and
a piezoelectric stack assembly coupled to the cutting assembly and configured to:
actuate the cutting assembly to cut vitreous of an eye.

2. The vitrectomy handpiece of claim 1, wherein the piezoelectric stack assembly comprises a plurality of piezoelectric disks.

3. The vitrectomy handpiece of claim 2, wherein a distance travelled by an inner cutting member of the cutting assembly is controlled by activating one or more of the plurality of piezoelectric disks.

4. The vitrectomy handpiece of claim 2, wherein the actuation of the cutting assembly is controlled by activating at least one of the plurality of piezoelectric disks.

5. The vitrectomy handpiece of claim 4, wherein activating the at least one of the plurality of piezoelectric disks comprises:
applying a voltage to the at least one of the plurality of piezoelectric disks, wherein the voltage is configured to cause the at least one of the plurality of piezoelectric disks to deform from a static state.

6. The vitrectomy handpiece of claim 5, wherein the deformation of the at least one of the plurality of piezoelectric disks causes a shaft to move in a distal direction, thereby causing an inner cutting member of the cutting assembly to move in the distal direction in relation to an outer cutting member of the cutting assembly.

7. The vitrectomy handpiece of claim 5, wherein the deformation of the at least one of the plurality of piezoelectric disks from the static state causes the at least one of the plurality of piezoelectric disks extending in a distal direction, thereby causing an inner cutting member of the cutting assembly to move in the distal direction in relation to an outer cutting member of the cutting assembly.

8. The vitrectomy handpiece of claim 7, wherein discontinuing the voltage causes the at least one of the plurality of piezoelectric disks to recede back to the static state in a proximal direction, thereby causing the inner cutting member of the cutting assembly to move in the proximal direction in relation to the outer cutting member of the cutting assembly.

9. The vitrectomy handpiece of claim 1, wherein the actuation of the cutting assembly comprises:
modulating an inner cutting member of the cutting assembly using an eigenfrequency.

10. The vitrectomy handpiece of claim 1, wherein the actuation of the cutting assembly comprises:
moving an inner cutting member of the cutting assembly in a longitudinal oscillatory motion.

11. The vitrectomy handpiece of claim 1, wherein the actuation of the cutting assembly comprises:
moving an inner cutting member of the cutting assembly in a harmonic motion, and
wherein the harmonic motion comprises a motion that is simultaneously a longitudinal oscillatory motion and a rotational oscillatory motion.

12. The vitrectomy handpiece of claim 1, wherein the piezoelectric stack assembly is coupled to a horn with a plurality of helical slits configured to introduce a rotational oscillatory motion to the cutting assembly.

13. The vitrectomy handpiece of claim 1, wherein the piezoelectric stack assembly comprises a plurality of angular piezoelectric elements disposed circumferentially about a longitudinal axis of the vitrectomy handpiece.

14. The vitrectomy handpiece of claim 1, wherein the piezoelectric stack assembly is further configured to:
actuate the cutting assembly at a cut-rate greater than at least 100,000 cuts per minute (CPM).

15. The vitrectomy handpiece of claim 1, wherein the piezoelectric stack assembly comprises a plurality of piezoelectric cones.
